(19) United States
(12) Patent Application Publication
(10) Pub. No.: US 2005/0181032 A1
(43) Pub. Date: Aug. 18, 2005

(54) Metastable pharmaceutical compositions

(75) Inventors: Nina Frances Wilkins, Victoria (AU); Kathryn Traci-Jane Klose, Victoria (AU); Timothy Matthias Morgan, Victoria (AU); Barry Leonard Reed, Victoria (AU); Barrie Charles Finnin, Victoria (AU)

Correspondence Address: FOLEY AND LARDNER;SUITE 500, 3000 K STREET NW, WASHINGTON, DC, 20007, US

(73) Assignee: Acrux DDS Pty Ltd.

(21) Appl. No.: 11/019,542

(22) Filed: Dec. 23, 2004

Related U.S. Application Data
(63) Continuation of application No. PCT/AU03/00785, filed on Jun. 24, 2003.

(30) Foreign Application Priority Data
Jun. 25, 2002 (AU) PS3173

(51) Int. Cl. A61K 31/506; A61K 31/445; A61K 9/70

(52) U.S. Cl. 424/449; 514/252.15; 514/317; 514/295

(57) ABSTRACT

The present invention provides a pharmaceutical composition for transdermal delivery comprising; one or more physiologically active agents; and a volatile pharmaceutically acceptable solvent; and wherein the physiological active agent forms a metastable deposit upon evaporation of the volatile solvent.

### FIELD OF THE INVENTION

The present invention relates to compositions for the transdermal delivery of physiologically active agents, to uses of those compositions, and to methods for the transdermal delivery of physiologically active agents.

### BACKGROUND OF THE INVENTION

There is a constant need for methods for the safe and effective administration of physiologically active agents. For many medications it is important that the administration regime is as simple and non-invasive as possible in order to maintain a high level of compliance by a patient. Oral administration is one administration regime that is commonly used because it is a relatively simple regime to follow. However, the oral administration route is also complicated because of complications associated with gastrointestinal irritation and with drug metabolism in the liver.

Administration of physiologically active agents through the skin (‘transdermal drug deliver’) has received increased attention because it not only provides a relatively simple dosage regime but it also provides a relatively slow and controlled route for release of a physiologically active agent into the systemic circulation. However, transdermal drug delivery is complicated by the fact that the skin behaves as a natural barrier and therefore transport of agents through the skin is a complex mechanism.

Structurally, the skin consists of two principle parts, a relatively thin outermost layer (the ‘epidermis’) and a thicker inner region (the ‘dermis’). The outermost layer of the epidermis (the ‘stratum corneum’) consists of flattened dead cells which are filled with keratin. The region between the flattened dead cells of the stratum corneum is filled with lipids which form lamellar phases that are responsible for the natural barrier properties of the skin.

For effective transdermal delivery of a physiologically active agent that is applied to the surface of the skin (‘topical application’), the agent must be partitioned firstly from the vehicle into the stratum corneum, it must typically then be diffused within the stratum corneum before being partitioned from the stratum corneum to the viable epidermis and dermis and then into the bloodstream.

To maximise the concentration (thermodynamic activity) of the physiologically active agent in the vehicle it is common for the physiologically active agent to be present as a saturated solution or mass.

To overcome some of the problems with transdermal delivery that are associated with transport across the dermal layers (‘percutaneous absorption’), physiologically active agents are commonly formulated with incorporation of one or more drug penetration enhancers. For example, aqueous ethanol is commonly used as a vehicle in formulations for topical application and it is known that ethanol can act as a penetration enhancer that can increase the flux of an active agent across the skin due to a solvent drag effect (Berner et al., 1989, J. Pharm. Sci, 78(5), 402-406. Penetration enhancers are known to cause skin irritation in some individuals. In the present invention, ethanol is unlikely to act as a penetration enhancer since, due to the small quantity applied to the skin, the ethanol acts as an intermediary solvent which spreads the drug and enhancer over the skin. Since the drying time of the compositions in question is less than 2 minutes, and in vitro weight loss measurements have previously confirmed that the ethanol evaporates off pig skin with a surface temperature of 32° C. within 1 minute all that remains is the drug. The short exposure time, less than 2 minutes, of ethanol or similar lipid extracting solvents to the skin is unlikely to alter the barrier function of the skin (Abrams et al., 1993, J. Invest. Dermatol, 101, 609-613).

U.S. Pat. No. 6,444,234 combines particular solvents and solute modifiers with skin stabilisers in a transdermal delivery system to form a true solution and thus minimizing irritation to the skin. U.S. Pat. No. 6,299,900 describes the non-occlusive delivery of a physiologically active agent across the skin however, this composition relies on a penetration enhancer to achieve an increased flux. Compositions formulated without the use of a penetration enhancer are generally not effective enough to deliver significant amounts of the physiological active through the skin. Compositions containing alcohol and acetone vehicles have been used for topical delivery of antibiotics, as seen in U.S. Pat. No. 6,017,912, which describes the use of fluoroquinolone in the topical treatment of skin infections and inflammatory conditions. However, such compositions have inherently low permeabilities of the physiological active.

In order to overcome the low permeability, the physiological active must have a maximised thermodynamic activity. According to U.S. Pat. No. 6,528,094, the use of stable shaped particles is particularly well-suited to the fabrication of pharmaceutical formulations, particularly where sustained release and uniform bioavailability are desired. It has been shown that to achieve high permeation rates across the skin, the concentrations of the drug dissolved may need to be high such that it possesses a high tendency to crystallise. As a result transdermal patches are often thermodynamically unstable because the drug shows a tendency to recrystallise during storage (Xinghang et al., 1996, Int. J. Pharm., 142(1), 115-119 and Latsch et al., 2003, Eur. J. Pharm. & Biopharm., In Press, Corrected Proof).

The use of testosterone in a volatile solvent has been previously disclosed (Wester et al., 1976). However, these compositions which consisted of testosterone dissolved in neat acetone have a propensity to de-fat (remove lipid) the skin under the chronic exposure conditions and therefore suffer from the disadvantage of causing skin irritation as opposed to the compositions that are the subject of this invention.

The discussion of the background to the invention herein is included to explain the context of the invention. This is not to be taken as an admission that any of the material referred to was published, known or part of the common general knowledge in Australia or any other country as at the priority date of any of the claims.

### SUMMARY OF THE INVENTION

The present invention arises from the inventor's studies of volatile sprays and aerosols and in particular from the realisation that, for finite dose formulations, appreciable enhancement of percutaneous absorption can be attained from the in situ formation of a metastable deposit within the skin using a volatile vehicle, such as a spray or aerosol.

Previous studies with these type of pharmaceutical compositions have indicated that the rate and extent of partitioning into the skin is already quite efficient with or without added penetration enhancer (Morgan et al., 1998, J. Pharm. Sci, 87(10), 1213-1218). The present invention arises, at least in part, from the realisation that an increase in the percutaneous absorption of the physiologically active agent may be achieved by the deliberate formation of a metastable drug in situ that has a lower melting point than would be otherwise achieved from the range of crystalline polymorph(s) routinely supplied by the commercial manufacturer. This reduction in melting point translates to an increase in the diffusion of the drug across the epidermis and dermis and into the bloodstream. To put the invention into practice the present inventor's have found that some combinations of physiologically active agent and volatile solvent form a metastable solid in situ when they are applied topically.

Accordingly, the present invention provides a composition including:
one or more physiologically active agents; and
a volatile pharmaceutically acceptable solvent
   wherein the physiologically active agent forms a metastable deposit upon evaporation of the volatile solvent. The lower melting point metastable deposit arises, at least in part, from either a metastable pseudopolymorph such as an alcohol solvate, a metastable polymorph, a metastable amorphous solid, or a mixture of these. Typically the reduction in melting point (at atmospheric pressure) provided by the metastable deposit will be in the range of from 1 to 50° C. and preferably from 2 to 30° C.

Unlike a solid precipitate (e.g. salt derivative of a drug) or a high melting point crystalline polymorph, the metastable deposit is readily partitioned into the skin upon evaporation of the volatile solvent and rapidly diffuses across the stratum corneum. The formation of the higher melting point crystalline deposits in the skin leads to a higher propensity toward skin irritation and a decrease in percutaneous absorption efficiency (due to the need for greater energy to dissolve the crystal prior to diffusional transport).

The metastable deposit also has excellent skin feel and touch when in some instances it may be desirable to rub the metastable deposit into the skin of a human.

The composition of the invention is essentially free of penetration enhancers. Penetration enhancers have the effect of increasing skin permeability by reversibly damaging or altering the physiochemical nature of the stratum corneum to reduce its diffusion resistance. Penetration enhancers are generally lipophilic, non-volatile compounds with a molecular weight greater than 100 and a vapour pressure below 10 mm Hg at atmospheric pressure and normal skin temperature of 32° C.

In addition to providing improved percutaneous absorption efficiency, the composition of the invention may also provide lower irritancy than some other delivery systems such as benzyl alcohol sprays, because the irritating penetration enhancer is removed. Also, the composition of the present invention may avoid problems with crystallisation and/or supersaturation that are encountered with storage of existing type transdermal patches because the pharmaceutical compositions of this invention exist as stable, single phase solutions and hence have no crystalline nature (or polymorphic memory) during their pharmaceutical shelf life. Another advantage of the present invention is the rapid partitioning into the skin from these in-situ forming metastable compositions which means the skin can act as a natural crystalline inhibitor for the physiologically active agent, further improving the stability of the metastable deposit within the skin. This leads to consistent and reliable delivery profile of the physiologically active agent over the desired dosage interval, which is typically once every 24 hours.

The present invention also provides a method of delivering a metastable drug formulation to a host, the method including the steps of applying a topical spray composition containing one or more physiologically active agents, and a volatile pharmaceutically acceptable solvent to the skin of the host so that the volatile solvent evaporates to form a metastable deposit containing the active agent.

The present invention further provides a composition for spray application to the skin of a subject for transdermal administration of a physiologically active agent, the composition comprising:
(i) a physiologically active agent;
(ii) a volatile pharmaceutically acceptable solvent; and
(iii) a propellant, preferably a fluoro hydrocarbon;
   wherein the composition when applied to the skin as a spray forms a metastable deposit in the skin of the subject.

The invention also provides a spray device for transdermal administration of a physiologically active agent comprising a chamber for maintaining the composition under pressure, a mixture contained within the chamber comprising the physiologically active agent, a pharmaceutically acceptable solvent which is volatile when applied to skin and a propellant which is preferably maintained at least partially in liquid form under pressure within the chamber and valve means for providing delivery of the mixture and wherein the mixture provides a metastable deposit of physiologically active agent on the skin. The propellant is preferably a hydrofluorocarbon such as HFC-134a or HFC-227. HFC-134a is the most preferred propellant. The hydrofluorocarbon propellant is preferably from 15 to 50% by volume of the total pharmaceutical composition and more preferably from 20 to 40% by volume of the total composition.

The invention further provides the use of an active agent for manufacture of a medicament for treatment or prophylaxis disease in a subject wherein the composition includes a physiologically active agent and a volatile carrier and is topically applied to the skin of the subject for transdermal administration wherein the volatile solvent evaporates to form a metastable deposit containing the active agent.

In addition the metastable composition avoids the disadvantage of spray nozzle blockage seen with existing film-forming sprays or aerosols.

The invention also provides a composition as described above contained in a chamber wherein the propellent is HFC-134a. The volatile solvent and active will preferably provide a single phase solution of the active.

As used herein the term ‘amorphous’ means substantially non-crystalline. It will be appreciated that, unless specified otherwise, the term amorphous includes within its scope a phase that does display some crystallinity.

The combination of physiologically active agent and volatile solvent of the present invention is limited functionally to those that together form a metastable deposit with a melting point lower than that seen from the range of crystalline polymorph(s) routinely supplied by the commercial manufacturer or obtained from crystallisation from methanol or chloroform. For this reason it is preferred that the active agent is relatively non-volatile relative to the volatile solvent so that upon application of the composition to the skin of the host, only the volatile solvent evaporates at physiological temperatures.

In practice, it has been found that the physiologically active agent may selected from a range of potent, lipophilc, physiologically active agents with a molecular weight less than 600 Daltons and a melting point less than 200° C. with the list including apomorphine, fentanyl, ropinirole, rivastigmine, buspirone, rizatriptan, anticholinergics such as oxybutynin, tolterodine and darifenacin, testosterone, MENT (7-methyl-19-testosterone), ethyinyl estradiol, or an pharmaceutically acceptable salt or derivative of any one of the aforementioned.

The drug and solvent generally have the ability to intimately mix, such that the solvent will interact with the crystal face of the drug. Therefore, the drug will typically have a saturated solubility in the solvent of greater than or equal to 0.05% w/w, more preferably greater than or equal to 0.1% w/w.

Preferably the volatile solvent has a vapour pressure is above 35 mm Hg at atmospheric pressure and normal skin temperature of 32° C. The volatile solvent will preferably comprise a lower alcohol. More preferably the volatile solvent will comprise at least 60% by weight of the total volatile solvent component of lower alcohol.

Most preferably the volatile solvent component will consist essentially of one or more lower alcohols. The preferred lower alcohols are ethanol, isopropanol and mixture thereof.

Conveniently, the composition is a topical spray composition that contains the physiologically active agent and the volatile solvent and the method includes the step of spraying the composition onto the skin of the host to form the metastable deposit containing the physiologically active substance.

In each of the above cases the metastable deposit is preferably formed in the epidermis of the host.

### BRIEF DESCRIPTION OF THE FIGURES

In the accompanying figures:

FIG. 1 shows a plot of the cumulative amount of drug penetrating across the skin from the metastable composition compared to a saturated solution of drug.

FIG. 2 gives an example of base DSC profiles before and after evaporation from ethanol.

FIG. 3 shows a plot of the melting point of each model drug, before and after evaporation from a volatile solvent after a time period of 24 hours.

FIG. 4 shows a plot of the diffusion profile for fentanyl through human epidermis, after evaporation from ethanol and isopropyl alcohol.

### DETAILED DESCRIPTION OF THE INVENTION

A benefit of the present invention is that the composition is stable, which means that it is not prone to supersaturation or crystallisation during its pharmaceutical shelf life. This may be contrasted with transdermal patches in which crystallisation of the active agent has presented a problem in the past. Thus the composition of the present invention can be held in a primary container during the shelf life without encountering shelf-life problems of the prior art transdermal patches.

The composition of the present invention may contain from about 0.1% to about 10% of the physiologically active agent, and from about 85% to about 99.8% of the volatile solvent.

Optionally, the vehicle may have additional pharmaceutical excipients, for example gelling agents, such as CARBOPOL and cellulose derivatives.

The invention will now be described with reference to the following examples. It is to be understood that the examples are provided by way of illustration of the invention and that they are in no way limiting to the scope of the invention.

### EXAMPLE 1

### In situ Formation of Metastable Drug Deposits

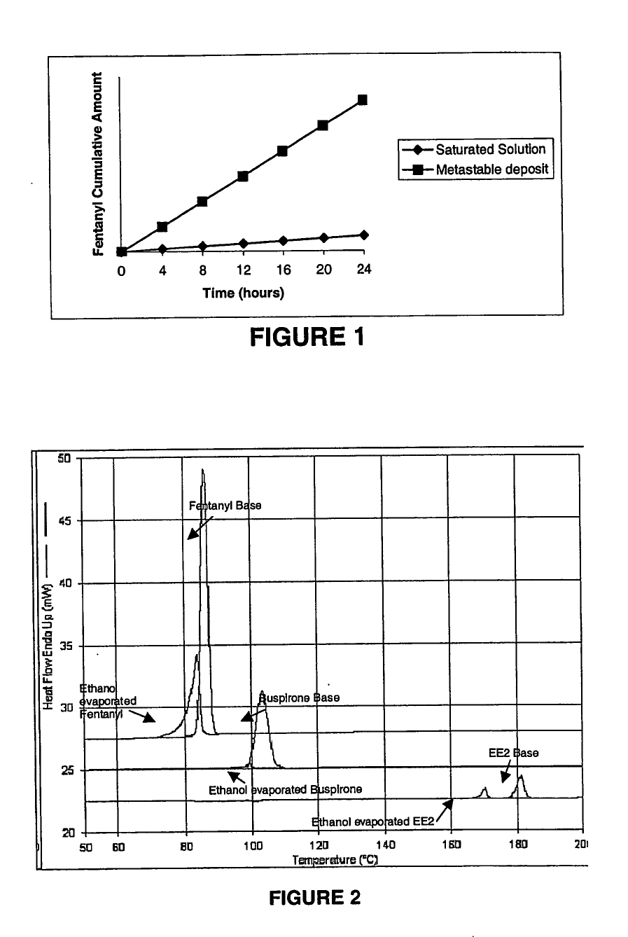

The formation of a metastable deposit of drug within the stratum corneum can be expected to increase the penetration of a drug across the skin relative to a saturated solution of a drug or a simple dispersion of the solid drug on the surface of the skin as shown in FIG. 1 (levels not detected).

### EXAMPLE 2

### Melting Point Reduction for Various Active Agents Mixed with Volatile Solvent

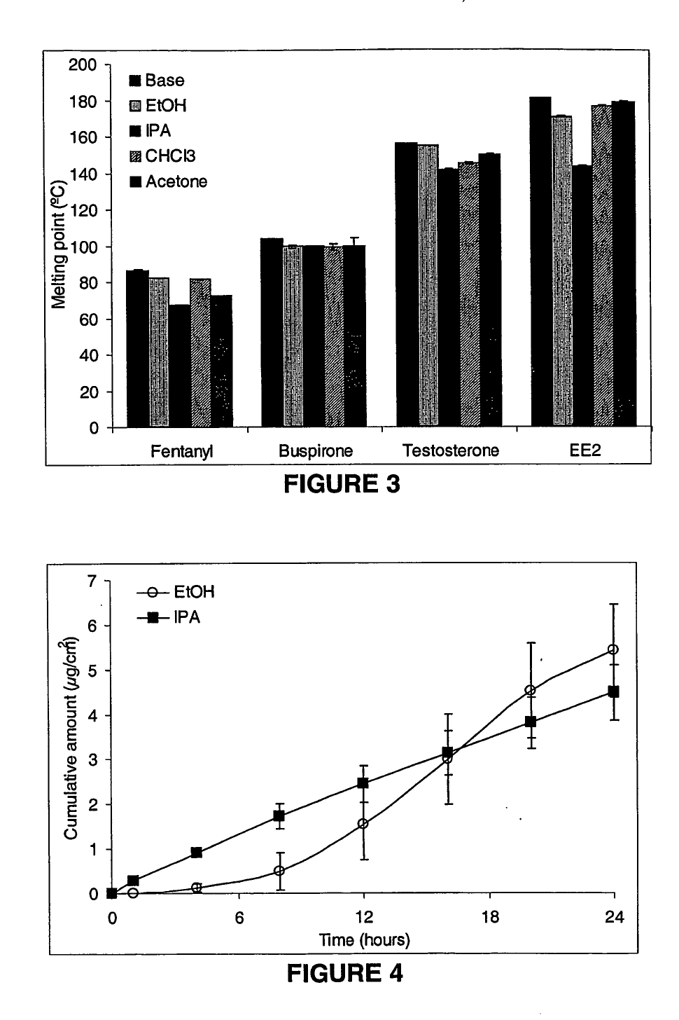

The ability to form a metastable deposit of the drug after evaporation of a volatile solvent can be determined by the reduction in melting point achieved 24 hours after solvent evaporation, as shown in FIGS. 2 and 3.

Materials
Fentanyl
Buspirone
Testosterone
Ethinyl Estradiol (EE2)
95% EtOH
Isopropyl alcohol (IPA)
Chloroform
Acetone

The active agents chosen represent a diverse range of physicochemical properties as shown in the following table:

Drugs of Choice—physiocochemical properties

| | MW Daltons | LogP | MP |
|---|---|---|---|
| Fentanyl | 336.48 | 3.79 | 84.0° C. |
| Buspirone | 385.51 | 2.63 | 103.5° C. |
| Testosterone | 288.4 | 3.01 | 156.0° C. |
| EE₂ | 296.4 | 3.91 | 180.8° C. |

Method

Various % w/v mixtures of active agent and volatile solvent were prepared. A 10 microlitre aluminium micro-DSC pan was then placed in a 50 microlitre aluminium DSC pan, and 5 microlitre aliquots of each formulation were pipetted into the 10 microlitre DSC pan. The volatile solvent was allowed to evaporate and further aliquots were re-applied until sufficient residue remained.

The pans were maintained at ambient temperature and 33% relative humidity for 24 hours. After this time the pans were covered and hermetically sealed. DSC was then performed under a stream of nitrogen and at 10° C./minute and within a temperature range that depended on the drug.

Results

Evaporation by each solvent resulted in melting point modulation (FIG. 2). DSC analysis of buspirone after solvent evaporation demonstrated the steadier melting point reduction (FIG. 3). In contrast, fentanyl, testosterone and EE2 showed very solvent dependent melting point reductions. In each case, isopropyl alcohol demonstrated the most significant difference in melting point change when compared to the compound base. Melting point depression is, we have found, an indication that the composition will have an increase in percutaneous absorption. In addition, the stratum corneum may prevent crystallisation, resulting in a solvent deposited metastable solid within the stratum corneum.

### EXAMPLE 3

### Effect of Metastable Compositions on Percutaneous Penetration

The choice of solvent used in a composition can be selected on the basis of the desired transdermal delivery profile as measured by percutaneous penetration (an example of which is shown in FIG. 4) in order to achieve the desired pharmacological effect.

The aforementioned examples are not meant to be limiting and it is envisaged that combinations of volatile solvents could also be used to obtain the desired pharmacological effect; for example on a weight basis.

| | |
|---|---|
| Ethanol:IPA | 20-80:20-80 |
| Ethanol or IPA:Acetone or Chloroform | 60-90:10-40; |

or a mixture thereof.

Diffusion Studies

Diffusion studies were conducted to determine the delivery profile of metastable compositions across skin.

Formulations
Fentanyl in ethanol (1 mole)
Fentanyl in isopropyl alcohol (1 mole)

All formulations were prepared by accurately weighing the appropriate amount of physiological active into a volumetric flask and made up to volume with appropriate volatile solvent.

Method

In vitro diffusion experiments were performed using stainless steel flow-through diffusion cells, using human epidermis maintained at 32° C. The receptor solution consisted of either 10% EtOH in 0.002% NaN3 or 20% EtOH in 0.002% NaN3. Eight cells for each condition were treated with 5 μl of appropriate donor phase, each a finite dose that was formulation dependent. Samples were collected at appropriate time points and analysed by high performance liquid chromatography (HPLC).

TABLE 1
HPLC conditions for receptor solution analysis.

| | Method | | | |
|---|---|---|---|---|
| Parameters | Buspirone | Fentanyl | | |
| Column | Symmetry C18 (3.9 * 150 mm) 5 μm | Symmetry C18 | | |
| | | (3.9 * 150 mm) | | |
| | | 5 μm | | |
| Mobile | Line A: 20% AcN in 0.01M | Line A: 5 nM TEA | | |
| Phase | KH2PO4 @ pH 2.85 nM | (milli-Q), pH 10.9 | | |
| | Line B: 90% AcN @ pH 2.8 | Line B: 100% AcN | | |
| | | Gradient: | | |
| Pump | Isocratic: | Time | % A | % B |
| | 70% A | 0 | 80 | 20 |
| | 30% B | 8.5 | 63 | 37 |
| | | 9 | 80 | 20 |
| | | 11 | 80 | 20 |

| | | |
|---|---|---|
| Flow rate | 1.0 ml/min | 1.0 ml/min |
| Absorbance | 239 nm | 210 nm |
| Injection | 50 μl | 50 μl |
| volume | | |
| Column | 40° C. | — |
| Temp. | | |

Results

Diffusion experiments were performed using fentanyl as a model compound, after ethanol and isopropyl alcohol evaporation.

Fentanyl diffusion profile, through human epidermis, demonstrates a change in delivery profile which is solvent dependent (FIG. 5). After evaporation from ethanol, fentanyl showed a sigmoidal, first order diffusion profile indicating that the initial burst across the skin is limited. However, after evaporation from isopropyl alcohol, fentanyl shows a zero order release rate profile. Therefore, the leaving tendency may be modified to suit the desired delivery rate by altering the volatile solvent vehicle.

1. A pharmaceutical composition for transdermal delivery comprising;
one or more physiologically active agents; and
a volatile pharmaceutically acceptable solvent;
wherein the composition is substantially free of non-volatile penetration enhancers and wherein the physiological active agent and the volatile solvent provide a metastable deposit comprising the pharmaceutical active upon evaporation of the volatile solvent.

2. A pharmaceutical composition for transdermal delivery comprising;
one or more physiological active compound selected from the group consisting of apomorphine, fentanyl, ropinirole, rivastigmine, buspirone, rizatriptan, anticholinergics, ethinyl estradiol or a pharmaceutically acceptable salt or derivative of any one of the aforementioned; and
a volatile pharmaceutically acceptable solvent;
wherein the composition is substantially free of non-volatile penetration enhancers and wherein the physiological active agent and the volatile solvent provide a metastable deposit comprising the pharmaceutical active upon evaporation of the volatile solvent.

3. A pharmaceutical composition according to claim 1 wherein the carrier comprises a hydrofluorocarbon propellant wherein topical application of the composition as an aerosol provides a metastable deposit on evaporation of the volatile carrier.

4. (canceled)

5. A pharmaceutical composition according to claim 3 wherein the propellent is HFC-134a.

6. A pharmaceutical composition according to claim 3 wherein the hydrofluorocarbon propellant is from 15 to 50% by volume of the total pharmaceutical composition.

7. A pharmaceutical composition according to claim 1 wherein the composition is contained in a chamber of a spray applicator device comprising a valve for delivering the composition from the chamber, a nozzle for dispersing the composition as an aerosol and means for providing a metered dose of aerosol from the nozzle said composition being retained under pressure within the chamber so as to maintain said propellent in a liquid form.

8. A pharmaceutical composition according to claim 1 wherein the volatile solvent and propellant provide a single phase solution of the active agent.

9. A pharmaceutical composition according to claim 1 wherein the composition comprises from 0.1% to 10% of physiologically active agent; and from 85% to 99.8% by weight of volatile solvent and propellant.

10. A pharmaceutical composition according to claim 1 wherein the physiologically active agent has a saturated solubility in the volatile solvent of not less than 0.05%.

11. A pharmaceutical composition according to claim 1 wherein the physiologically active agent component comprise a molecular weight of less than 600 Daltons and a melting point less than 200° C.

12. A pharmaceutical composition according to claim 1 wherein the physiologically active agent comprises one or more compounds selected from the group consisting of apomorphine, fentanyl, ropinirole, rivastigmine, buspirone, rizatriptan, ethinyl estradiol or a pharmaceutically acceptable salt or derivative thereof.

13. A pharmaceutical composition according to claim 1 wherein the physiologically active agent comprises of testosterone, MENT (7-methyl-19-testosterone), or a pharmaceutically acceptable salt or derivative thereof.

14. A pharmaceutical composition according to claim 1 wherein the volatile solvent has a vapour pressure above 35 mmHg at atmospheric pressure and a temperature of 32° C.

15. A pharmaceutical composition according to claim 1 wherein the volatile solvent comprises one or more lower alcohols.

16. A pharmaceutical composition according to claim 1 wherein at least 60% by weight of the volatile solvent comprises one or more lower alcohols.

17. A pharmaceutical composition according to claim 1 wherein the volatile solvent consisting essentially of one or more lower alkanols.

18. A pharmaceutical composition according to claim 16 wherein the lower alcohols are selected from ethanol and isopropanol.

19. A pharmaceutical composition according to claim 1 wherein a thickening agent is present from 0.1% to 20% by weight of the total pharmaceutical composition.

20. A pharmaceutical composition according to claim 19 wherein the thickening agent is a cellulose derivative, more preferably hydroxypropyl cellulose, hydroxypropyl-methyl cellulose, hydroxyethyl cellulose, or a mixture thereof.

21. A pharmaceutical composition according to claim 19 wherein the thickening agent is CARBOPOL.

22. A method of treatment to provide enhanced percutaneous absorption of a physiologically active substance, the method comprising applying a spray of a pharmaceutical composition according to claim 1 to the skin of a subject to form a metastable deposit of the active agent upon evaporation of the volatile solvent whereby petitionary of the physiologically active agent from the stratum corneum to the viable epidermis is enhanced.
